(19) United States
(12) Patent Application Publication
(10) Pub. No.: US 2026/0174017 A1
(43) Pub. Date: Jun. 25, 2026

(54) INTEGRATED SYSTEM FOR COMPREHENSIVE COASTAL ECOSYSTEM MONITORING, DISEASE PREDICTION, AND ENVIRONMENTAL ASSESSMENT

(71) Applicant: Abu Dhabi Maritime Academy SOLE PROPRIETORSHIP LLC, Abu Dhabi (AE)

(72) Inventors: Fawzeya HAIMDI, Abu Dhabi (AE); Dmitry MIKHAYLOV, Abu Dhabi (AE); Muhammad SAEED, Abu Dhabi (AE); Mohamed ALHOSANI, Abu Dhabi (AE)

(21) Appl. No.: 18/988,452

(22) Filed: Dec. 19, 2024

Related U.S. Application Data
(60) Provisional application No. 63/545,778, filed on Oct. 26, 2023.

(51) Int. Cl. A01G 7/06; A01G 23/00; A01K 61/00; G01N 33/00; G06F 17/18; G06Q 10/0637; G06Q 50/02

(52) U.S. Cl. CPC A01G 7/06; A01G 23/00; A01K 61/00; G01N 33/0098; G06F 17/18

(57) ABSTRACT

This patent presents an integrated system for monitoring coastal ecosystems, focusing on mangroves and corals in Gulf region-specific environments. It utilizes a Multi-Faceted Neural Network (MFNN) to process diverse data sources, offering insights into mangrove health, carbon sequestration potential, coral health, disease spread prediction, and site selection for mangrove restoration. The system employs various training and optimization techniques to ensure precision and adaptability, including backpropagation, regularization, fine-tuning with regional datasets, and data augmentation. It introduces mathematical formulas for evaluating mangrove canopies and coral ecosystems, addressing disease prediction and optimal planting site selection for mangrove restoration. The system's interconnection with carbon storage research enhances its scientific foundation for assessing overall carbon storage in mangrove ecosystems. This system and its associated methods provide a powerful toolset for coastal ecosystem monitoring, preservation, and restoration, promoting sustainable management practices.

### BACKGROUND

Mangrove ecosystems stand as invaluable natural treasures due to their multifaceted contributions to our environment. Beyond their aesthetic beauty, they serve as carbon sequestration powerhouses, diligently storing atmospheric carbon dioxide (CO2) and playing a pivotal role as biogeochemical filters, maintaining the equilibrium of coastal ecosystems. The importance of mangroves extends to their function as critical habitat for diverse flora and fauna, including numerous commercially important fish species.

The significance of mangroves, however, has long been under threat from various factors, including urbanization, climate change, and anthropogenic activities. Accurate monitoring of mangroves has become imperative, not only to assess their health but also to quantify their carbon storage capacity (CSC) and the environmental benefits they offer.

Corals, while distinct from mangroves, play a crucial role in the overall health of coastal ecosystems. They provide essential habitat for various marine species, including those that contribute to the well-being of mangroves. Therefore, monitoring both mangroves and corals is vital to understanding and preserving the health of these interconnected ecosystems.

The spread of diseases can occur due to factors such as wind patterns and deforestation. Monitoring and predicting these factors are essential for preserving coastal ecosystems' health.

### Solution

This patent introduces an innovative and integrated system dedicated to the meticulous monitoring of mangroves and corals thriving in Gulf region-specific ecosystems. This state-of-the-art system incorporates modules designed to analyze mangrove homogeneity, create carbon polygons, assess and verify carbon levels, compute environmental credits, predict the spread of diseases, and select optimal planting sites for mangroves based on fresh water sources.

### Multi-Faceted Neural Network (MFNN) Architecture

At the heart of this revolutionary system lies the Multi-Faceted Neural Network (MFNN), a neural architecture of remarkable sophistication, painstakingly designed to handle the rich diversity of data sources inherent to mangrove and coral ecosystems. The MFNN is a formidable tool specifically fine-tuned to provide profound insights into mangrove health, carbon sequestration potential, coral health, disease spread prediction, and site selection based on fresh water sources.

### MFNN Architecture Overview:

1. Data Input Layers: The MFNN boasts an exceptional capability to ingest data from a multitude of sources. These sources encompass LiDAR-generated 3D models of mangrove canopies, spectral data procured from remote sensors, environmental variables, and ground-based measurements for both mangroves and corals.
2. Parallel Processing Branches: The MFNN employs dedicated parallel processing branches, each tailored to address diverse data types. For LiDAR-generated 3D models, convolutional layers and attention mechanisms are masterfully deployed to discern meaningful features connected to the structure of mangrove canopies and coral formations.
3. Recurrent Layers: Recurrent layers, a cornerstone of the MFNN architecture, are strategically woven into the fabric of this neural network. They are instrumental in dissecting temporal data, unearthing patterns over time. This capability is particularly valuable for scrutinizing environmental variables, ground-based measurements, and the health of dynamic coral populations.
4. Fusion Layers: In the final act of data transformation, outputs from parallel branches and recurrent layers elegantly converge within specialized fusion layers. This convergence creates a comprehensive and holistic representation of mangrove health, carbon storage potential, coral well-being, and disease spread patterns, ensuring no nuances escape our scrutiny.

### Training and Optimization:

To ensure the MFNN's capabilities are honed to perfection, it embarks on an arduous journey of training and optimization. This rigorous process is indispensable to establish the intricate relationships between diverse data inputs and the indicators of mangrove health, coral health, disease spread, and site suitability.
1. Backpropagation and Optimization Techniques: The MFNN is nurtured through backpropagation, ably supported by optimization techniques such as the Adam optimizer. These mechanisms allow the neural network to adjust its parameters, continuously fine-tuning its understanding of the complexities of mangrove and coral ecosystems.
2. Regularization Methods: To prevent overfitting and maintain the model's generalizability, regularization methods like dropout are judiciously applied during the training process.
3. Fine-tuning with Regional Datasets: Recognizing the unique characteristics of Gulf region mangrove and coral ecosystems, the MFNN undergoes specialized fine-tuning. This step ensures that the neural network remains adaptable and responsive to regional variations, ultimately enhancing its precision.
4. Data Augmentation: Data augmentation techniques are deployed to augment the MFNN's dataset, bolstering its resilience and capacity to handle diverse scenarios and regional nuances.

### Key Indicators and Formulas:

### Mangrove Evaluation:

1. Mangrove Canopy Density (MCD):
Formula: MCD=(Number of trees in a defined area)/(Total area)
2. Canopy Cover Percentage (CCP):
Formula: CCP=(Area covered by mangrove canopies)/(Total area)×100%
3. Chlorophyll Content Index (CCI):
Formula: CCI=(Total chlorophyll content)/(Number of mangrove trees)

### Coral Evaluation:

1. Coral Health Index (CHI):
Formula: CHI=(Total number of healthy corals)/(Total number of corals)×100%
2. Coral Growth Rate (CGR):
Formula: CGR=(Change in coral size)/(Initial coral size)/(Time)
3. Coral Biodiversity Index (CBI):
Formula: CBI=(Number of coral species)/(Total number of corals)

### Disease Prediction:

Utilizes data on factors such as wind patterns and tree cutting to predict the spread of diseases in mangrove forests.

### Site Selection for Planting Mangroves:

Identifies optimal locations based on sources of fresh water, precipitation patterns, groundwater availability, and proximity to freshwater bodies.
   Interconnection with Carbon Storage Research:

Based on these data, Chinese scientists have explored potential correlations between Aboveground Carbon (AGC) and Belowground Carbon (BGC) in mangrove forests, aiming to provide a scientific foundation for assessing the overall carbon storage in mangrove forests.

1. An integrated system for monitoring and predicting the health of mangroves and corals, disease spread, and optimal planting site selection, employing a Multi-Faceted Neural Network (MFNN) architecture for comprehensive data analysis.
Mathematical Details:
The MFNN processes diverse data sources, including LiDAR-generated 3D models (M_mangrove, M_coral), spectral data (S_mangrove, S_coral), environmental variables (E_mangrove, E_coral), and ground-based measurements (G_mangrove, G_coral), using convolutional layers and attention mechanisms to extract meaningful features for comprehensive analysis.

2. The system of claim 1, wherein the MFNN processes diverse data sources, including LiDAR-generated 3D models, spectral data, environmental variables, and ground-based measurements.

3. A method for evaluating mangrove canopies, including calculating Mangrove Canopy Density (MCD), Canopy Cover Percentage (CCP), and Chlorophyll Content Index (CCI), to assess mangrove health and carbon sequestration potential.
MCD=(Number of trees in a defined area)/(Total area)
CCP=(Area covered by mangrove canopies)/(Total area)×100%
CCI=(Total chlorophyll content)/(Number of mangrove trees)

4. A method for evaluating corals, including determining Coral Health Index (CHI), Coral Growth Rate (CGR), and Coral Biodiversity Index (CBI) to assess coral health and biodiversity.
CHI=(Total number of healthy corals)/(Total number of corals)×100%
CGR=(Change in coral size)/(Initial coral size)/(Time)
CBI=(Number of coral species)/(Total number of corals)

5. A method for assessing the spatial and temporal variability of greenhouse gas emissions and absorption, as well as determining integral values of fluxes for different territories over defined time intervals, within the largest and most significant mangrove biogeocenoses, using carbon polygons developed on these biogeocenoses.
Mathematical Details:
Integrating data from carbon polygons, calculate greenhouse gas emissions (GHG_e) and absorption (GHG_a) over time intervals and territories, considering variations in AGC and BGC: GHG_e=∫[t1, t2](∫[Area]AGC_t−AGC_t−1 dArea)dt
GHG_a=∫[t1,t2](∫[Area]BGC_t−BGC_t−1dArea)dt

6. A method for developing and perfecting instrumental measurement methods by scientific researchers within the established carbon polygons, aimed at assessing the emission and absorption of greenhouse gases, with a focus on spatial and temporal variability, and determining integral values of fluxes for various territories within defined time intervals.
Mathematical Details:
Utilizing ground-based measurements, the method employs statistical analysis to optimize measurement instruments and procedures. It utilizes data from carbon polygons to estimate greenhouse gas fluxes (GF) with spatial and temporal variation:
GF=∫[t1,t2](∫[Area](GHG_e−GHG_a)dArea)dt
Where GHG_e represents greenhouse gas emissions, GHG_a represents greenhouse gas absorption, and the integral is computed over specific time intervals and areas.

7. A method for designing and adapting technologies for remote monitoring of the structure and condition of vegetation and soil (subaqueous) cover, greenhouse gas emissions, and absorption using data from ground-based measurements and mathematical modelling methods, specifically tailored for mangrove ecosystems.
Mathematical Details:
The method involves the development of mathematical models (M_model) that incorporate data from ground-based measurements (GBM) to monitor mangrove ecosystems remotely. The models are optimized to estimate parameters related to vegetation and soil conditions and greenhouse gas fluxes:
M_model=f(GBM)
Where f represents the mathematical function relating ground-based measurements to the model's parameters.

8. A method for conducting experiments to identify the most significant and high-productivity carbon-sequestering plant species, their distribution, and the restoration of anthropogenically disrupted biogeocenoses within mangrove ecosystems, further contributing to carbon sequestration.
Mathematical Details:
Experimental assessments involve quantifying the growth rates (GR) of various plant species (S_i) over time, considering their distribution within the mangrove ecosystem:
GR(S_i)=(Change in biomass of S_i)/(Initial biomass of S_i)/(Time)
The method employs statistical analysis to determine the most efficient carbon-sequestering plant species for restoration efforts.

9. A method for selecting rational resource utilization methods for mangrove forests and regenerative management in permissible areas within mangrove biogeocenoses, promoting sustainable mangrove forest management and carbon sequestration.
This method utilizes optimization algorithms to identify rational resource utilization methods (RRUM) that maximize carbon sequestration while ensuring sustainable mangrove forest management. It considers factors such as growth rates (GR), biomass, and carbon storage potential (CSP) of mangrove stands: RRUM=argmax {GR, Biomass, CSP}
Where argmax represents the argument that maximizes the selected criteria.

10. A method for predicting the spread of diseases in mangrove forests based on factors such as wind patterns and tree cutting, facilitating early intervention and disease control.
Mathematical Details:
Disease spread prediction employs mathematical modelling to estimate disease propagation rates (DPR) based on factors like wind patterns (WP) and deforestation rates (DR):
DPR=f(WP,DR)
The method calculates the risk of disease outbreaks, aiding in early intervention strategies.

11. A method for identifying optimal planting sites for mangroves based on comprehensive assessments of fresh water sources, precipitation patterns, groundwater availability, and proximity to freshwater bodies, enhancing the success of mangrove restoration and conservation efforts.
Mathematical Details:
Optimal planting site selection involves mathematical modelling to assess factors such as precipitation patterns (PP), groundwater availability (GA), proximity to freshwater bodies (PFB), and the presence of natural fresh water sources (FWS). The method aims to maximize mangrove growth potential:
Optimal Site=argmax{PP,GA,PFB,FWS}
Where argmax represents the argument that maximizes the selected criteria for site suitability.
